(19) United States
(12) Patent Application Publication
(10) Pub. No.: US 2009/0093701 A1
(43) Pub. Date: Apr. 9, 2009

(54) METHOD AND APPARATUS FOR SCANNING A SUBJECT

(76) Inventors: Kevin Scott Hakl, Oxford (GB); Veit Ulrich Boris Schenk, Mullheim (DE); Thomas George Wright, Oxford (GB)

Correspondence Address: SCHIFF HARDIN, LLP;PATENT DEPARTMENT, 6600 SEARS TOWER, CHICAGO, IL, 60606-6473, US

(21) Appl. No.: 12/205,972

(22) Filed: Sep. 8, 2008

(30) Foreign Application Priority Data
Sep. 7, 2007 (GB) 0717418.8

(51) Int. Cl. A61B 5/05

(52) U.S. Cl. 600/407

(57) ABSTRACT

In a patient scanner and method, a scan image is registered with a reference image and superimposed. In an iterative process the images are normalized and the transparency varied to assist in the identification of abnormalities.

### BACKGROUND OF THE INVENTION

1. Field of the Invention

This invention relates to a scanning apparatus and method for scanning a subject, such as a patient, particularly but not exclusively for performing a positron emission tomography (PET) scan.

2. Description of the Prior Art

In positron emission tomography scanning, a patient has a radioactive pharmachemical injected which is taken up into the body's organs, for example, the brain, and emits therefrom radioactivity which is detected by a number of detectors arranged in a detecting ring or doughnut. By careful processing it is possible to derive from the detected emissions images of the organ which may be used for diagnosis of a patient's condition.

The images captured in this way may be used to identify tumors and other abnormalities due to their different “take up” of the radio emitter. However, the patient's responses to the pharmachemical may vary due, in particular, stress levels caused by an unfamiliar and daunting procedure. Other factors will also affect the response. These will include the food the patient has eaten, the time delay between administration of the pharmachemical and the ambient temperature which affects brown fat absorption. The factors will affect the scan in different ways making the analysis of the image and diagnosis difficult.

### SUMMARY OF THE INVENTION

An object of the present invention is to alleviate problems.

According to the invention there is provided apparatus for scanning a patient comprising a scanner for obtaining scan data representative of part of the patient being scanned, an image reconstructor for reconstructing an image using the data, and an adjustment unit that varies (changes) at least one parameter of at least one of the image and reference image such that the relative significance of at least some parts of the image and the reference image is changed.

The invention also encompasses a method implemented by such an apparatus.

Preferably, the at least one parameter varied is image transparency. When the transparency of one of the images is changed relative to the other areas having the same image intensity will remain the same. This will have the effect of those parts of the images remaining visually the same while other parts of the image will become less apparent.

Preferably, after the at least one parameter is varied an at least second parameter is varied to equalize the scans. The preferred second parameter is image intensity, to normalize the images. Image intensity may be a property of brilliance, contrast or hue but in the preferred embodiment the property is image brilliance. This may be varied over part or all of the image, for example, over a region of interest.

The reference image may be an image that will enhance the detection of certain conditions and may be an image of a healthy patient or indeed a patient having a certain condition. Then the scanned image and the reference image may be varied to enhance the diagnosis of the patient's condition when the images are displayed.

In the specific embodiment, the adjustment unit that varies the at least one parameter is configured to change the property of transparency of at least one of the image and the reference image. A display at which the image and the reference image are presented can be provided.

Preferably, the adjustment unit that varies the at least one parameter is configured to vary the relative visual significance of the displayed image and reference image. While this may be done without user interaction using image analysis software, preferably the adjustment unit varies the at least one parameter in response to a user input. The user input may be made via a mouse, keyboard or touch screen or other user input device.

In a specific embodiment the scanner is a positron emission tomography scanner but the invention is applicable to other types of scanners. For example, SPECT or magnetic resonance. The patient may be a human or animal.

In the described embodiment the reference and the scan image are displayed on a display unit and a user varies the images to cause a merging of normal parts of the scan raising the relative significance of abnormal parts of the scan, that is to say, parts which depart from the reference “normal”.

### BRIEF DESCRIPTION OF THE DRAWINGS

FIG. 1 schematically illustrates an apparatus for scanning a patient in accordance with the invention;

FIG. 2 shows a patient scan and a reference image being merged to highlight a point of interest in the patient scan.

FIG. 3 is a flowchart showing an embodiment of the inventive method.

### DESCRIPTION OF THE PREFERRED EMBODIMENTS

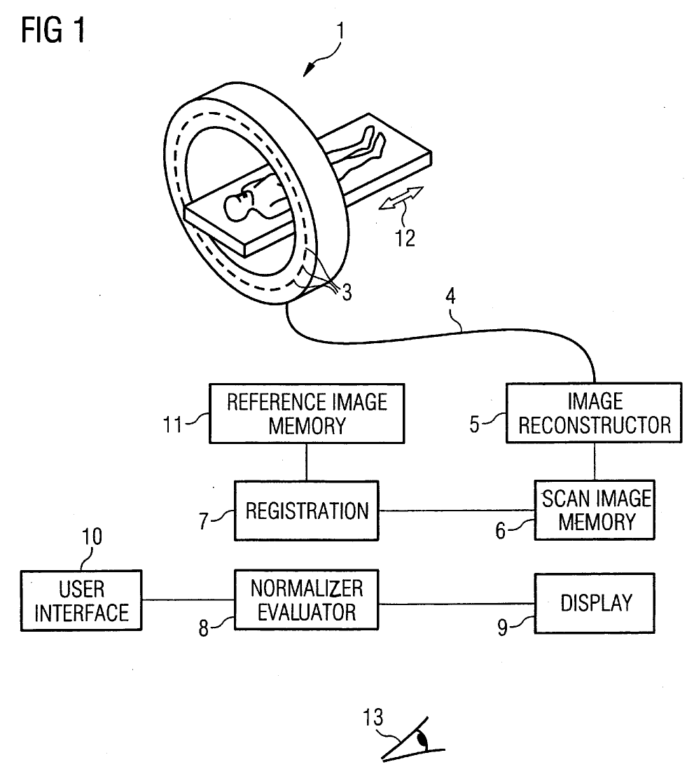

As is shown in FIG. 1, a patient scanning apparatus 1 has a scanning ring 2 which includes a number of detectors 3 providing outputs to a data bus 4. An image reconstructor 5 is connected to the other end of the data bus 4. The apparatus also includes a scan image memory 6, a registration processor 7, an evaluator and normalizer 8, a display 9, a user interface 10 and a reference memory 11. The patient is loaded onto a bed which is moved along the axis of the scanning ring to various so-called bed positions, as indicated by arrow 12, by a motor (not shown).

The scanner is in this case a positron emission tomography (PET) scanner (but other scanners may be used.) This requires the administration of a radio pharmachemical to a patient and the elapse of a period of time for its uniform absorption. The chemical emits positrons which are detected by the detectors 3 as the patient is moved through the various bed positions.

The detector outputs are used to reconstruct images by the image reconstructor 5 for every bed position, which are stored in the memory 6. This is well known to those skilled in the art. The memory 6 is coupled to the registration processor 7 to allow the downloading of the images. The registration processor 7 carries out a registration process to align the images with a set of reference images held in memory 11 to form a registered pair. The registered pairs of images are passed to the normalizer 8 which displays the pairs on the display 9. A user 13 adjusts the images in this embodiment using the user interface 10. (This may also be done by a microprocessor performing an image analysis.)

The manner of adjustment in this case is iterative involving a normalization of the images and a “check”. In the check, the relative transparency of the images is varied in a manner to be later described to enable a determination to be made of how well registered the images are. It is important to note that other ways of varying the images may be possible. For example, color or contrast may be varied.

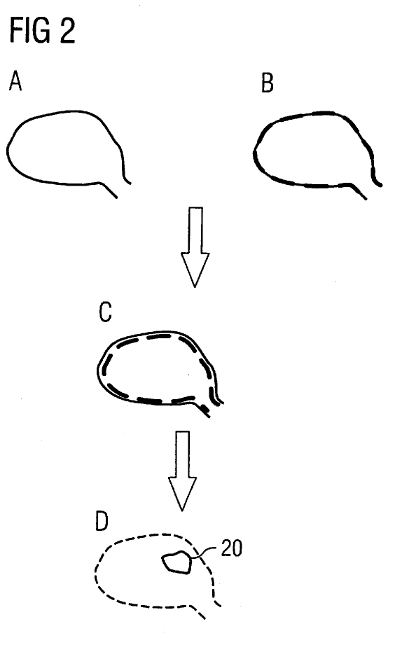

FIG. 2 depicts the registration of the reference and the scanned image in broad terms. Image A is a scan through a body organ in this case a patient's brain. Image B is the reference image. The registration processor 7 registers these images and passes them as an image pair to the evaluator and normalizer 8. The evaluator and normalizer 8 displays them one on top of the other on the display 9 to provide a combined image C. The user 13 then performs the iterative process of normalization and checking using a mouse of the user interface 10.

In the iterative process the user varies the relative intensity, in this case brilliance, of the images to render them substantially normalized at a region of interest. This may be done by varying the brilliance of one or both of the images to create the relative change. An apparent abnormality 20 is then discernable by a user.

In the check, the transparency of the images is varied. The result of this variation in transparency will tend to make more apparent those features which exhibit greatest variation, and those which have the least variation, until the similarities between the images are enhanced as depicted by image D.

The reference image and the scanned image may also be shown side by side rather than superimposed one on top of the other.

In an alternative embodiment of the invention, the evaluator and normalizer 8 may take the part of the user 13 by processing the images, and measuring a property such as brilliance in the images or in parts of the images (an alternative may be intensity). The brilliance value may be stored and one of the component images varied. The resultant image brilliance may be compared with the earlier ones stored and varied again until the minimum/maximum value reached. The combined image that gives the minimum/maximum brilliance value is then displayed on the display 9. Again other image parameters such as color may be utilized.

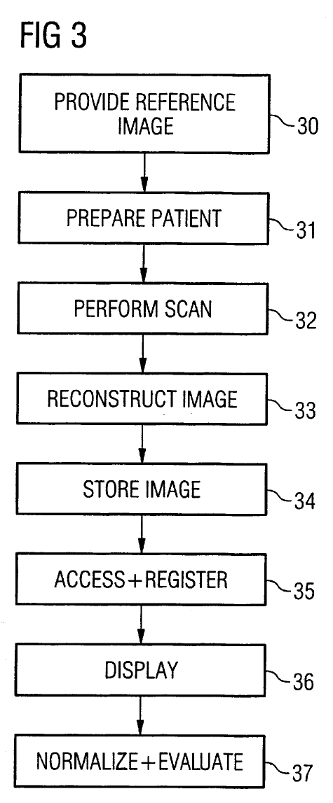

Thus, the steps of the method for utilizing the invention comprises, as is shown in FIG. 3, a first step, step 30, to provide a suitable reference. This is loaded into the reference memory 11. This step may also involve the selection of a reference from a number of references and it may be made by a selection of a particular scan type.

In a second step, step 31, the patient is prepared. This involves the administration of a pharmachemical, its subsequent absorption and the positioning of the patient on the bed. In step 32 the scan is carried out by moving the patient through the bed positions 12.

Each of the bed positions results in a number of images which are reconstructed from the detector data in step 33 and stored in step 34 in memory 6. The memory 6 is accessed and the images registered with the reference images in step 35 by the registration processor 7.

In step 36 the image pairs are displayed on the display 9.

In the next step, step 37, the images are normalized by varying the relative brilliance over the image or a point of interest to identify abnormalities and also checked by varying the transparency. This involves the user interacting via the user interface 10 with the evaluator and normalizer 8.

Although further modifications and changes may be suggested by those skilled in the art, it is the intention of the inventors to embody within the patent warranted hereon all changes and modifications as reasonably and properly come within the scope of their contribution to the art.

We claim as our invention:

1. An apparatus for scanning a patient comprising a scanner for obtaining scan data representative of part of the patient being scanned, an image reconstructor for reconstructing an image using the data, and an adjustment unit configured to vary at least one parameter of at least one of the image and reference image such that the relative significance of at least some parts of the image and the reference image is changed.

2. An apparatus as claimed in claim 1 wherein the adjustment unit comprises means to change a property of transparency of at least one of the image and the reference image.

3. An apparatus as claimed in claim 1 comprising a display that displays the image and the reference image.

4. An apparatus as claimed in claim in claim 3 wherein the adjustment unit is configured to vary, as the at least one parameter, the relative visual significance of the displayed image and reference image.

5. An apparatus as claimed in claim 4 comprising a user interface and wherein the adjustment unit is configured to vary the at least one parameter in response to an input made via said user interface.

6. An apparatus as claimed in claim 1 comprising a further adjustment unit configured to vary a second property of at least one of the image and reference image.

7. An apparatus as claimed in claim 6 wherein the further adjustment unit is configured to vary image intensity as said second property.

8. An apparatus as claimed in claim 1 wherein the scanner is a positron emission tomography scanner.

9. A method for scanning a patient comprising:
providing a reference image scanning the patient to produce a patient image;
changing at least one parameter of at least one of the reference and the patient image to change a relative visual significance to provide a changed image or images; and
outputting at least the changed image or images.

10. A method as claimed in claim 9 wherein the patient image and reference image are registered before the at least parameter are changed and the changed image or images used to determine a measure of the registration.

11. A method as claimed in claim 10 wherein the images are displayed to a user.

12. A method as claimed in claim 11 wherein the images are displayed superimposed.

13. A method as claimed in claim 9 where the parameter changed is the transparency of the image or images.

14. A method as claimed in claim 1 comprising changing a second parameter of the image or images to normalize the images or parts of the images.

15. A method as claimed in claim 14 wherein the second parameter is image intensity.

16. A method as claimed in claim 15 wherein the image intensity is varied by varying image brilliance.
